(19) United States
(12) Patent Application Publication
(10) Pub. No.: US 2009/0048304 A1
(43) Pub. Date: Feb. 19, 2009

(54) Crystal Form of Besipirdine Chlorhydrate, Process Preparation and Use Thereof

(75) Inventors: Hugues Bienayme, St. Symphorien D'Ozon (FR); Jacques Ferte, Issy-Les-Moulineaux (FR)

Correspondence Address: OLIFF & BERRIDGE, PLC, P.O. BOX 320850, ALEXANDRIA, VA, 22320-4850, US

(73) Assignee: UROGENE, Paris (FR)

(21) Appl. No.: 12/223,602

(22) PCT Filed: Feb. 20, 2007
(86) PCT No.: PCT/IB2007/001456

§ 371 Date: Sep. 22, 2008

Related U.S. Application Data
(60) Provisional application No. 60/787,157, filed on Mar. 30, 2006.

(30) Foreign Application Priority Data
Feb. 20, 2006 (FR) 06 01468

(51) Int. Cl. C07D 401/12; A61K 31/4439; A61P 13/10

(52) U.S. Cl. 514/339; 546/277.4

(57) ABSTRACT

Crystal form of besipirdine chlorhydrate (Form I) corresponding to the Formula (A) below: the aforementioned form being characterized at least by one of the following physico-chemical properties: a) In FTIR, it displays at least the following absorption bands of the infrared spectrum: 778, 1198, 1121, but not the following absorption bands of the infrared spectrum: 3395, 1583, 732, the aforementioned bands being expressed in cm⁻¹ at ±5 cm⁻¹; b) In PXRD, it shows at least the following reflections, which are the most intense ones but whose intensity hereafter is given for information only: Formula (II) c) In DSC, it displays at least an endothermic peak at 187.3±2.0° C. using 5° C./min scanning conditions, and a fusion enthalpy ΔH of 130.4±2.0 J/g. The invention also relates to the processes used for the preparation of form I as well as its applications in urology.

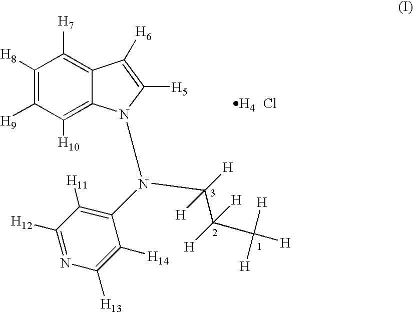

The present invention relates to a stable crystal form, called form I, of N-propyl-N-(4-pyridinyl)-1H-indol-1-amine chlorhydrate (or besipirdine HCl), to its characterisation, to the processes used for obtaining it and to its applications, more particularly in the pharmaceutical field.

N-propyl-N-(4-pyridinyl)-1H-indol-1-amine or besipirdine, represented in its chlorhydrate form by the formula A below, belongs to the N-(4-pyridinyl)-1H-indol-1-amine family.

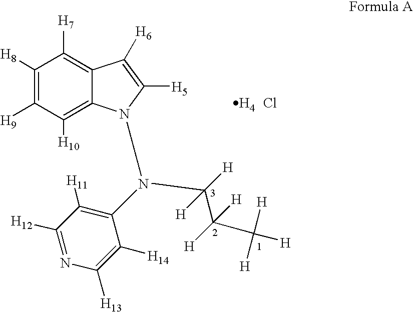

In the description hereafter, the term “besipirdine” is equally used to refer to besipirdine and its salts; the expression “besipirdine.HCl” strictly refers to besipirdine chlorydrate.

U.S. Pat. No. 4,970,218, WO02/064126 and WO2005/035496 documents describe the obtention of N-(4-pyridinyl)-1H-indol-1-amines, these processes make it possible to prepare besipirdine and its salts. According to WO02/064126 document, it is known that some N-(4-pyridinyl)-1H-indol-1-amines can have pharmaceutical applications; for example, N-(3-fluoro-4-pyridinyl)-N-propyl-3-methyl-1H-indol-1-amine (or HP184) was shown to slightly decrease the frequency of bladder contractions induced by bladder irritation in vivo, in rats.

The applicant recently found that besipirdine can be used in the treatment of symptoms associated with bladder irritation or related to effort incontinence and mixed incontinence.

In view of the prevalence of these disorders, besipirdine appears of great interest in pharmacy.

The present invention is based on the discovery that besipirdine.HCl exists as several different crystal forms differing from each other in stability, in particular. The besipirdine synthesis processes described at present lead to a compound whose polymorphic profile is not reproducible from one batch to another. Hence different batches can contain different polymorphs in variable proportions. Moreover, the polymorphic profile of certain batches synthesised using these methods has been shown to change with time, over a several months period.

This lack of reproducibility and stability of the polymorphic profile over time makes it impossible to precisely control the pharmaceutical quality of the active compound. It can also influence the properties of final products containing this compound. Therefore identifying the stable form of besipirdine.HCl and determining the process(es) allowing its reproducible obtention appears highly desirable.

Three polymorphic forms, called I, II, III, and two solvates called IV (methanol solvate) and V (ethanol solvate) of besipirdine.HCl were identified that are obtained as a mixture after the synthesis processes described above. Form II is the most predominant form obtained following on from the process described in WO2005/035496, in which it is isolated from conventional techniques; more precisely, according to the exemplified method of synthesis, the product is obtained by precipitation of besipirdine solution in its base form using methanolic chlorhydric acid in n-butyl acetate solution.

These five forms were characterized and the form I, i.e. the stable form of besipirdine.HCl which, once obtained, does not evolve with time in storage conditions at room temperature, was characterized and compared with the other crystal and solvate forms II, III, IV and V.

Hence the present invention relates to a crystal form of besipirdine.HCl, called form I, corresponding to the formula A above and characterized by at least one of the following physico-chemical properties:

a) In FTIR, form I displays at least the following absorption bands of the infrared spectrum: 778, 1198, 1121, but not the following absorption bands of the infrared spectrum: 3395, 1583, 732, the aforementioned bands being expressed in cm⁻¹ at ±5 cm⁻¹;

b) In PXRD, diffractogram of form I shows at least the following reflections, which are the most intense ones but whose intensity hereafter is given for information only:

| | Angle (2θ) | | | | | | |
|---|---|---|---|---|---|---|---|
| | 12.61 | 14.11 | 18.98 | 19.93 | 21.03 | 25.13 | 25.91 |
| Intensity* (%) | 77.13 | 71.82 | 73.00 | 67.21 | 61.08 | 60.44 | 100.00 |

*for information only

c) In DSC, form I displays at least an endothermic peak at 187.3±2.0° C. using 5° C./min scanning conditions, and a fusion enthalpy ΔH of 130.4±2.0 J/g.

Thanks to at least one of the characteristics mentioned above, form I can be distinguished from each one of the other forms II, III, IV and V. It is preferentially characterized by at least two of the characteristics a), b) and c) above, if not all of them.

The experimental conditions in which these physicochemical characteristics were measured are precisely described hereafter in dedicated subsections.

By RMN ¹H, besipirdine.HCl is characterized by a spectrum recorded in deutered chloroform (CDCl₃) using a Bruker 200 MHz instrument and presents the peaks reported in table 1 below, the proton numbering being the one used in formula A herebefore.

TABLE 1

| | Chemical displacement |
|---|---|
| Protons | δH (ppm) |
| H4 | 16.32 (br.s) |
| H12, H13 | 8.22 (d) |
| H7 | 7.62 (d) |
| H8, H9 | 7.17 (d) |
| H5 | 7.06 (d) |
| H10 | 7.02 (d) |
| H6 | 6.62 (d) |
| H11, H14 | 6.59 (br.s) |
| H3 | 4.00 (m) |
| H2 | 1.76 (m) |
| H1 | 0.99 (t) |

The characterisation of crystal form I of interest according to the invention is explained hereafter. To this aim, each of the other forms II, III, IV and V was also identified and this characterisation is also part of the present invention.

1) Infra-Red Spectroscopy

IR spectroscopy was performed using a FTIR (Fourier Transform Infrared Spectroscopy) spectrometer Bruker IFS 113V, between 4000 and 600 cm⁻¹, using diamond Attenuated Total Reflectance (ATR).

Sample of each one of besipirdine.HCl crystal forms I, II, III, IV and V was prepared without compression.

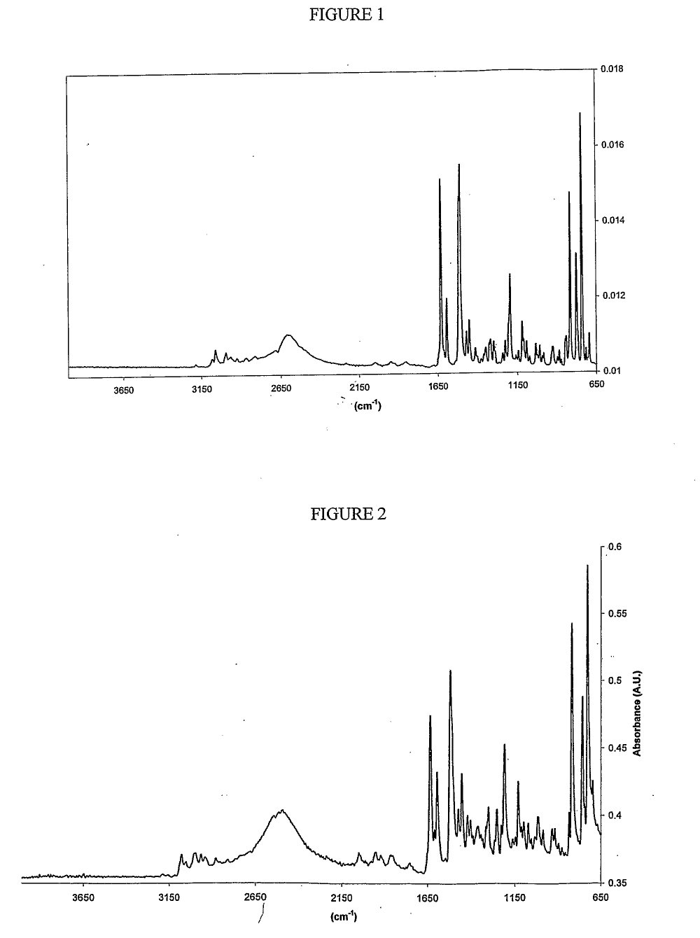

Spectra obtained are shown in FIGS. 1 to 5 and are characterized by the absorption bands reported in table 2.

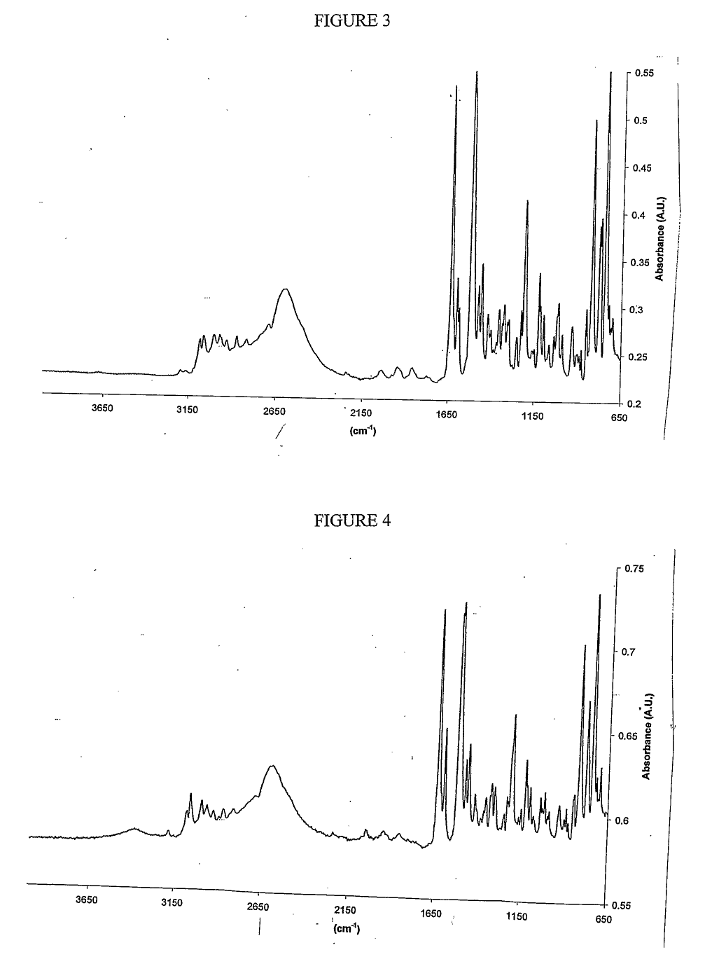

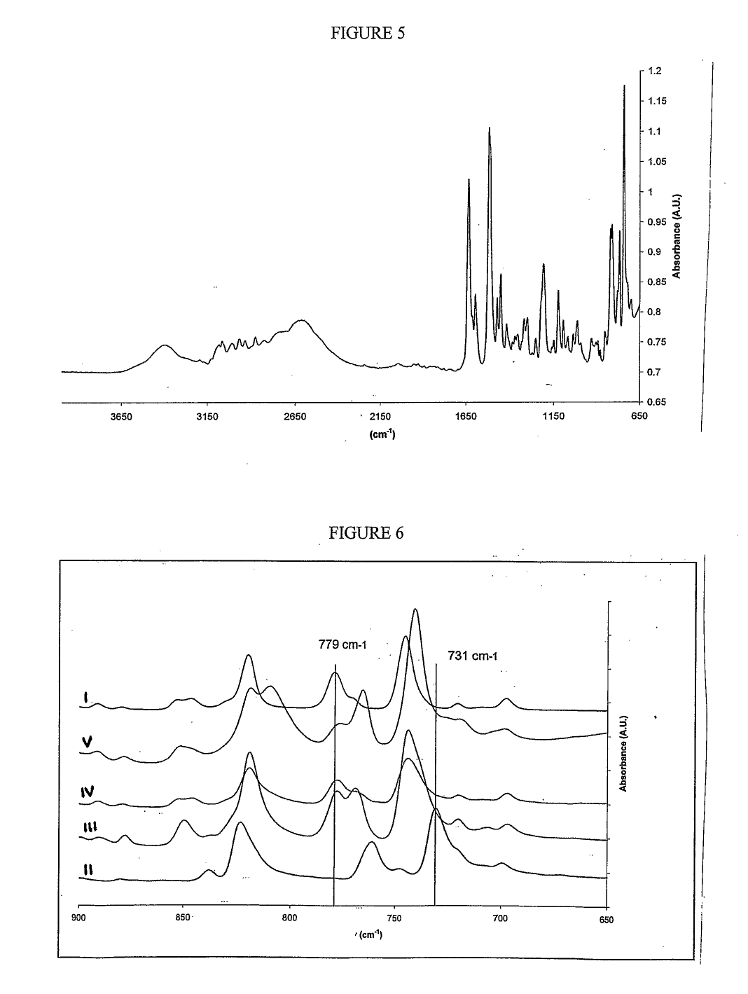

TABLE 2
λ (cm⁻¹)

| Form I | Form II | Form III | Form IV | Form V |
|---|---|---|---|---|
| (FIG. 1) | (FIG. 2) | (FIG. 3) | (FIG. 4) | (FIG. 5) |
| 3188 | 3191 | 3195 | 3395 | 3395 |
| 3080 | 3158 | 3163 | 3191 | 3196 |
| 3062 | 3117 | 3084 | 3155 | 3168 |
| 2999 | 3075 | 3061 | 3086 | 3084 |
| 2967 | 3051 | 3001 | 3059 | 3065 |
| 2917 | 3001 | 2973 | 2996 | 3005 |
| 2868 | 2963 | 2931 | 2968 | 2968 |
| 2814 | 2940 | 2870 | 2926 | 2936 |
| 2684 | 2880 | 2815 | 2870 | 2870 |
| 2611 | 2805 | 2689 | 2815 | 2824 |
| 2048 | 2675 | 2601 | 2685 | 2708 |
| 1945 | 2546 | 2244 | 2610 | 2615 |
| 1859 | 2517 | 2039 | 2048 | 2244 |
| 1631 | 2494 | 1942 | 1951 | 2044 |
| 1593 | 2048 | 1858 | 1853 | 1951 |
| 1510 | 1951 | 1770 | 1631 | 1923 |
| 1471 | 1923 | 1634 | 1595 | 1892 |
| 1454 | 1863 | 1598 | 1509 | 1858 |
| 1416 | 1756 | 1583 | 1472 | 1774 |
| 1406 | 1636 | 1517 | 1453 | 1737 |
| 1380 | 1610 | 1470 | 1417 | 1634 |
| 1364 | 1595 | 1453 | 1405 | 1595 |
| 1350 | 1519 | 1415 | 1382 | 1518 |
| 1327 | 1474 | 1405 | 1364 | 1472 |
| 1321 | 1453 | 1398 | 1352 | 1451 |
| 1301 | 1420 | 1347 | 1325 | 1417 |
| 1293 | 1402 | 1318 | 1320 | 1383 |
| 1243 | 1390 | 1298 | 1300 | 1368 |
| 1230 | 1362 | 1291 | 1294 | 1354 |
| 1198 | 1345 | 1246 | 1244 | 1316 |
| 1160 | 1335 | 1222 | 1229 | 1296 |
| 1146 | 1313 | 1203 | 1197 | 1250 |
| 1121 | 1301 | 1159 | 1161 | 1220 |
| 1111 | 1265 | 1148 | 1146 | 1207 |
| 1095 | 1252 | 1122 | 1122 | 1161 |
| 1078 | 1227 | 1113 | 1113 | 1148 |
| 1036 | 1209 | 1095 | 1095 | 1122 |
| 1011 | 1163 | 1063 | 1078 | 1068 |
| 998 | 1148 | 1036 | 1038 | 1036 |
| 990 | 1131 | 1012 | 1011 | 1012 |
| 930 | 1112 | 987 | 997 | 993 |
| 904 | 1100 | 930 | 988 | 927 |
| 892 | 1072 | 902 | 930 | 902 |
| 879 | 1055 | 889 | 902 | 892 |
| 848 | 1033 | 875 | 890 | 879 |
| 820 | 1014 | 849 | 879 | 851 |
| 778 | 919 | 819 | 847 | 817 |
| 745 | 898 | 779 | 819 | 809 |
| 720 | 879 | 769 | 777 | 775 |
| 697 | 860 | 743 | 744 | 766 |
| | 838 | 720 | 720 | 741 |
| | 824 | 707 | 697 | 720 |
| | 760 | 697 | | 699 |
| | 749 | | | |
| | 732 | | | |
| | 699 | | | |
| | 671 | | | |

FITR analysis shows that forms I, II, III, IV and V have different absorption frequencies, indicating that these forms have different crystal structures. In particular, a large band at 3395 cm⁻¹ can be attributed to a hydroxyle group and allows for the characterisation of solvates IV et V. The displacement of these bands from their position in ethanol or methanol spectra indicates the presence of hydrogene bonds.

Therefore, in order to define form I, the following profile was used that combines the presence and the absence of absorption bands of the infrared spectrum: presence of bands 778, 1198, 1121 and absence of bands 3395, 1583, 732, the aforementioned bands being expressed in cm⁻¹ at ±5 cm⁻¹.

Generally speaking, the FITR spectrum of form I looks similar to that of FIG. 1, obtained in aforementioned conditions.

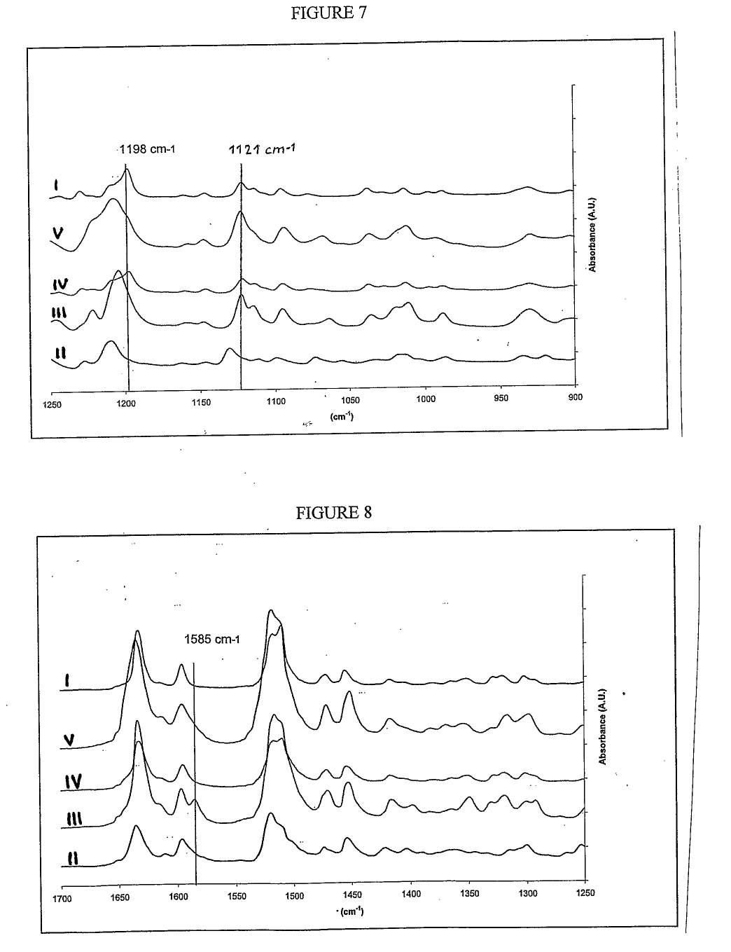

FIGS. 6, 7 and 8 show stacking of forms I to V spectra at determined wavelength intervals: FIG. 6 for 900-650 cm⁻¹ range, FIG. 7 for 250-900 cm⁻¹ range, and FIG. 8 for 1700-1250 cm⁻¹ range.

2) X-Ray Diffraction

X-ray crystallography was performed on each of the forms I to V as a powder (Powder XRD). This technique allows the identification of crystal forms and solvates. Diffractograms were recorded using a Philips X'pert Pro diffractometer equipped with a copper anticathode (wavelength λ=1,54051 Å) et d'un générateur (I=20 mA; U=40 kV).

Measures were performed on a diffraction scale angles ranging from 2 to 60°2θ with a 0.03°2θ pitch.

Each sample was put on a glass slide without any prior grinding.

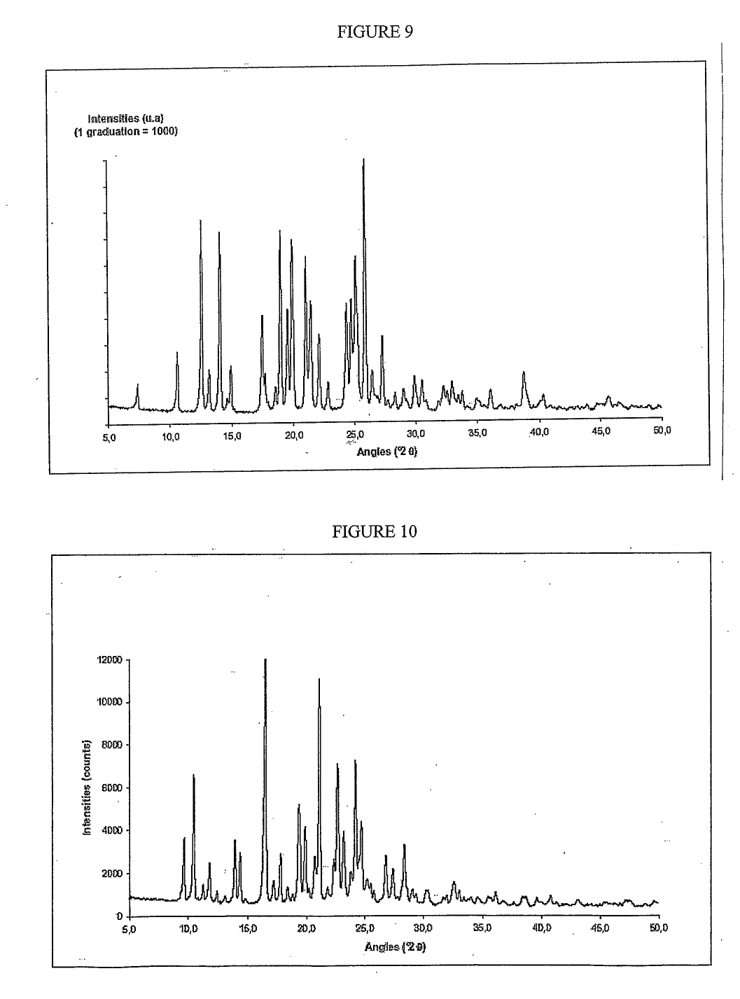

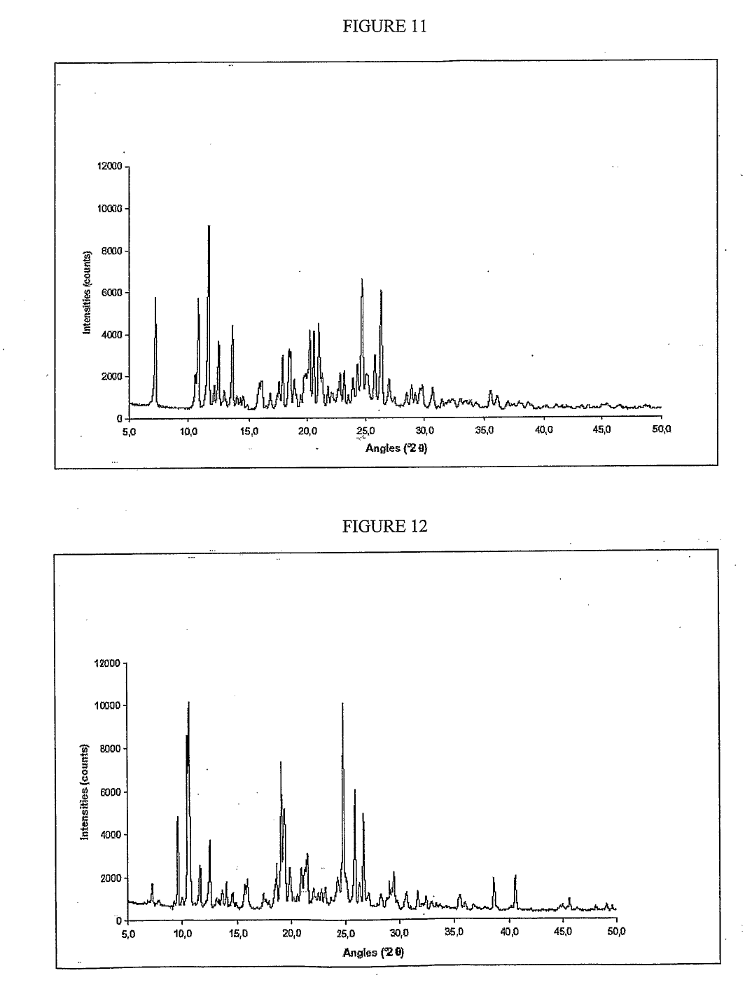

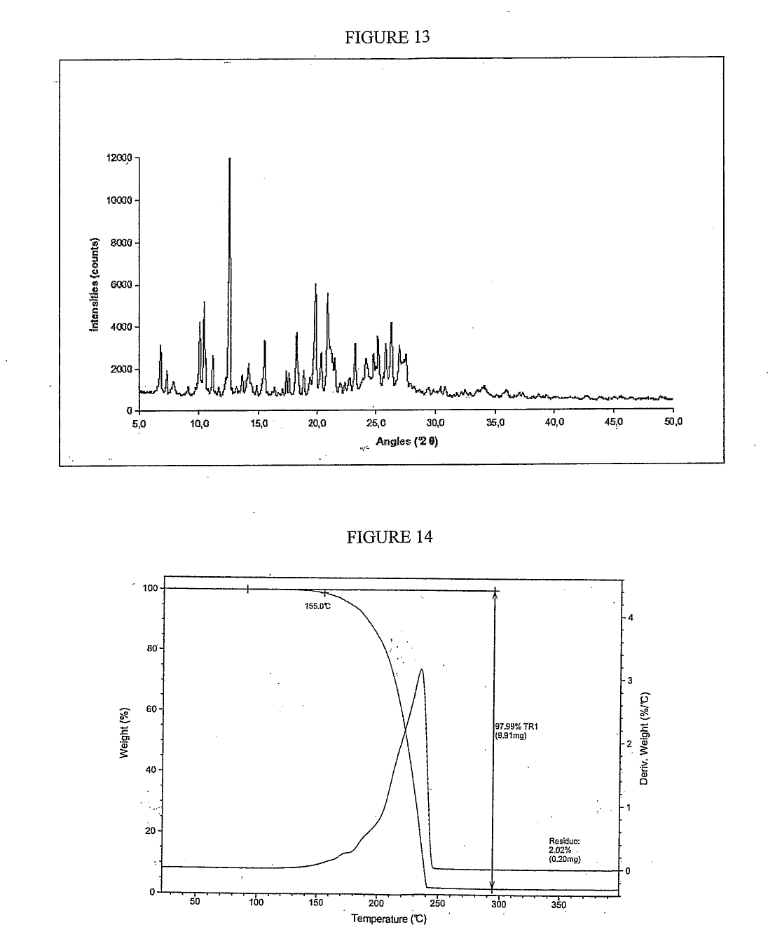

The diffractograms obtained are shown in FIGS. 9, 10, 11, 12 and 13 for forms I, II, III, IV and V, respectively, and the most intense diffraction peaks, characteristic of each form, are reported in table 3, together with the aspect and the chemical purity of each of forms I to V.

TABLE 3

| | | Chemical | PXRD* | |
|---|---|---|---|---|
| | | purity** | Characteristic | |
| Forms | Aspect | (%) | peaks (2θ) | Intensity (%) |
| I | White | 99.13 | 12.61-14.11- | 77.13-71.82- |
| | powder | | 18.98-19.93-21.03- | 73.00-67.21- |
| | | | 25.13-25.91 | 61.08-60.44- |
| | | | | 100.00 |
| II | Brown | 100 | 10.54-16.54- | 49.71-100.00- |
| | powder | | 21.18-22.71-24.25 | 86.30-53.57- |
| | | | | 55.16 |
| III | Beige | 100 | 7.25-10.84-11.68- | 59.55-58.65- |
| | powder | | 13.66-24.71-26.34 | 100.00-45.14- |
| | | | | 68.00-61.23 |
| IV | Translucent | 100 | 10.51-10.70- | 83.95-100.00- |
| | crystal | | 19.05-24.88-25.92 | 70.44-95.65- |
| | | | | 54.46 |
| V | Translucent | 95.45 | 10.05-10.42- | 10.28-11.90- |
| | crystal | | 12.54-19.89-20.93 | 100.00-15.18- |
| | | | | 13.61 |

*Peaks correspondig to 100% are underlined.
**Determined by HPLC

For each form, the most intense peak, normalised to 100%, is characteristic and allows to distinguish the different forms from each other. Hence the most intense reflections (indicated in table 3 above) were selected for defining form I according to the invention.

Generally speaking, powder diffractogram of form I looks similar to that of FIG. 6, obtained in aforementioned conditions.

3) Structure Determination from Powder Diagram

Crystal structure of form I was determined from its powder diffractogram using a range of software:
indexation software, Dicvol91: Boultif, A. et Louër, D., <<Indexing of powder diffraction patterns for low-symmetry lattices by the successive dichotomy method >>, J. Appl. Cryst., 24, 987-993, 1991,
Rietveld refining software: Petricek, V., Dusek, M. & Palatinus, L., 2000, Jana2000. The crystallographic computing system. Institute of Physics, Praha, Czech Republic,
and simulation software, ENDEAVOUR: H. Putz, J. C. Schön, M. Jansen, Combined Method for ‘Ab Initio’ Structure Solution from PowderDiffraction Data, J. Appl. Cryst. 32, 864-870, 1999.

The crystal parameters obtained using this methods are the following:

Monoclinical lattice

Space group P21/c

a=12.1698(7)Å, b=7.3815(4)Å, c=16.777(1)Å

α=90°, β=92.825(4)°, γ=90°

Z=2

Table 4 indicates the coordinates of carbon and nitrogen atoms in the crystal structure.

TABLE 4

| Atom | x | y | z | Uiso |
|---|---|---|---|---|
| Cl1 | 0.4520(10) | −0.5748(14) | 0.1141(7) | 0.069(6) |
| N1 (amine) | 0.765(3) | 0.233(4) | 0.141(2) | 0.032(13) |
| N2 | 0.567(2) | −0.211(4) | 0.105(2) | 0.060(16) |
| (pyridine) | | | | |
| N3 (indole) | 0.769(4) | 0.316(5) | 0.225(2) | 0.047(14) |
| C1 | 0.577(3) | −0.145(6) | 0.173(2) | 0.07(2) |
| (pyridine) | | | | |
| C2 (indole) | 0.724(3) | 0.497(4) | 0.310(2) | 0.028(15) |
| C3 (indole) | 0.829(4) | 0.435(6) | 0.333(3) | 0.039(17) |
| C4 (indole) | 0.873(5) | 0.326(6) | 0.268(3) | 0.05(2) |
| C5 | 0.610(3) | −0.127(5) | 0.043(2) | 0.06(2) |
| (pyridine) | | | | |
| C6 | 0.681(3) | 0.026(6) | 0.052(3) | 0.051(17) |
| (pyridine) | | | | |
| C7 (propyl) | 0.828(3) | 0.329(6) | 0.079(2) | 0.09(2) |
| C8 (propyl) | 0.773(3) | 0.465(5) | 0.036(2) | 0.10(2) |
| C9 (propyl) | 0.839(2) | 0.525(5) | −0.0397(18) | 0.098(18) |
| C10 (indole) | 0.699(3) | 0.445(5) | 0.240(2) | 0.07(2) |
| C11 (indole) | 0.964(4) | 0.228(5) | 0.269(2) | 0.05(2) |
| C12 (indole) | 0.915(4) | 0.454(4) | 0.390(2) | 0.029(16) |
| C13 | 0.688(4) | 0.085(6) | 0.130(3) | 0.027(15) |
| (pyridine) | | | | |
| C14 | 0.642(3) | 0.004(5) | 0.196(2) | 0.042(18) |
| (pyridine) | | | | |
| C15 (indole) | 1.045(3) | 0.271(5) | 0.329(3) | 0.07(2) |
| C16 (indole) | 1.013(4) | 0.380(5) | 0.384(2) | 0.029(16) |

FIG. 24 shows the diffractogram of form I powder that was used for the determination as well as the difference between the observed and the calculated diffractograms, the latter being represented by the lowest “trace”.

4) Thermogravimetry (TG)

Thermogravimetry involves monitoring the weight loss of a sample thermically induced, as a function of the applied temperature.

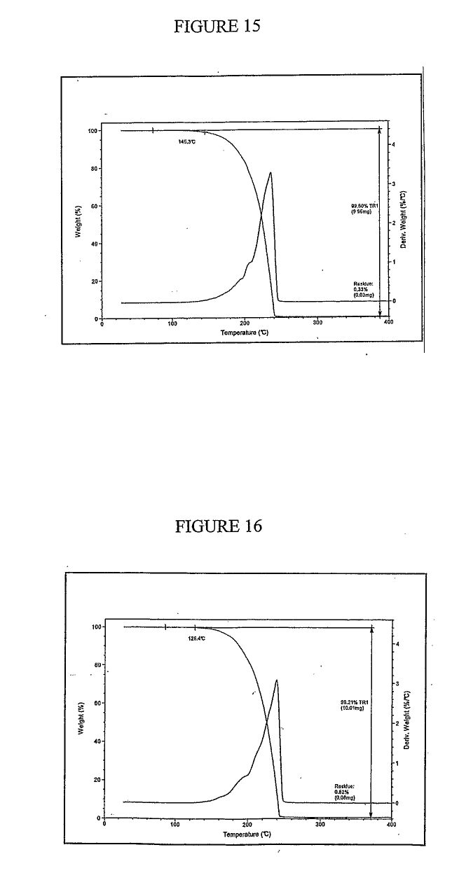

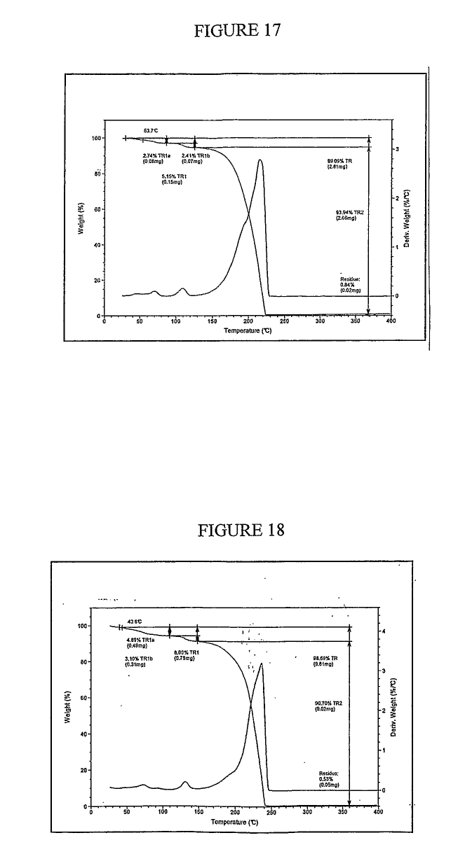

Thermogravimetric analyses were performed on a TA Instruments TGA 2950 instrument, with a 0.1 μg resolution over a scale ranging from 0 to 100 mg. Samples were placed under a nitrogen stream (60 mL/min) and heated at a 5° C./min speed over a temperature interval between 20 and 400° C. TG diagrams are represented in FIGS. 14, 15, 16, 17 et 18 corresponding to forms I, II, III, IV, and V, respectively.

Form II TG indicates a sublimation (and/or vaporisation) process from 145.3° C.

Form IV TG shows a weight loss between 53.7 and 125.4° C., attributed to a desolvatation process corresponding to 0.49 mols of solvent.

Form V TG shows a weight loss in two steps between 43.6 and 148.2° C., attributed to a desolvatation process corresponding to 0.55 mols of solvant.

5) Differential Scanning Calorimetry (DSC)

This technique measures the thermic flux (absorption/emission) response of a sample as a function of temperature and time.

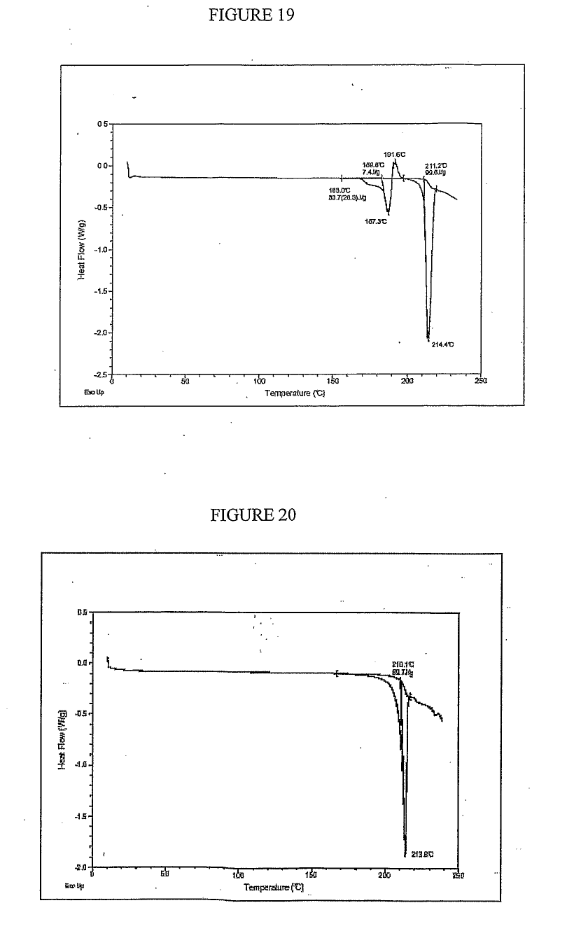

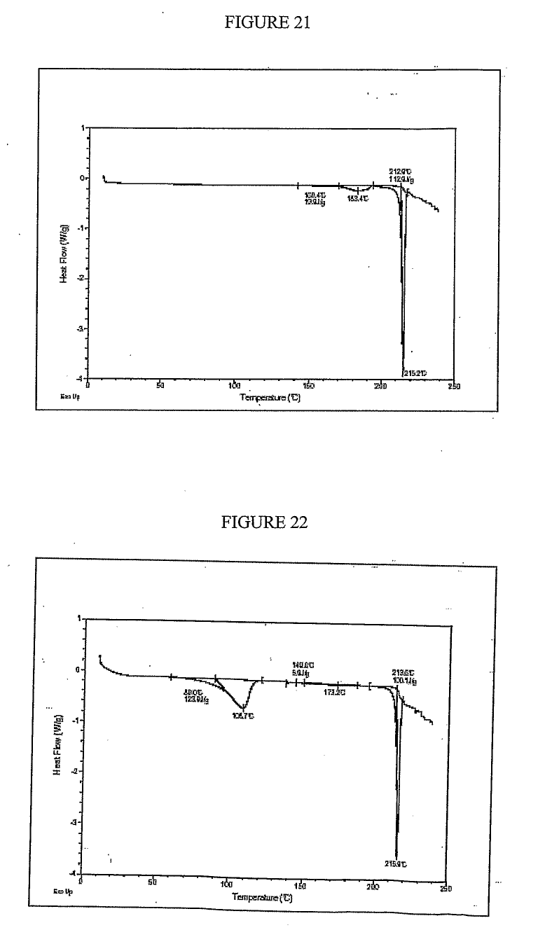

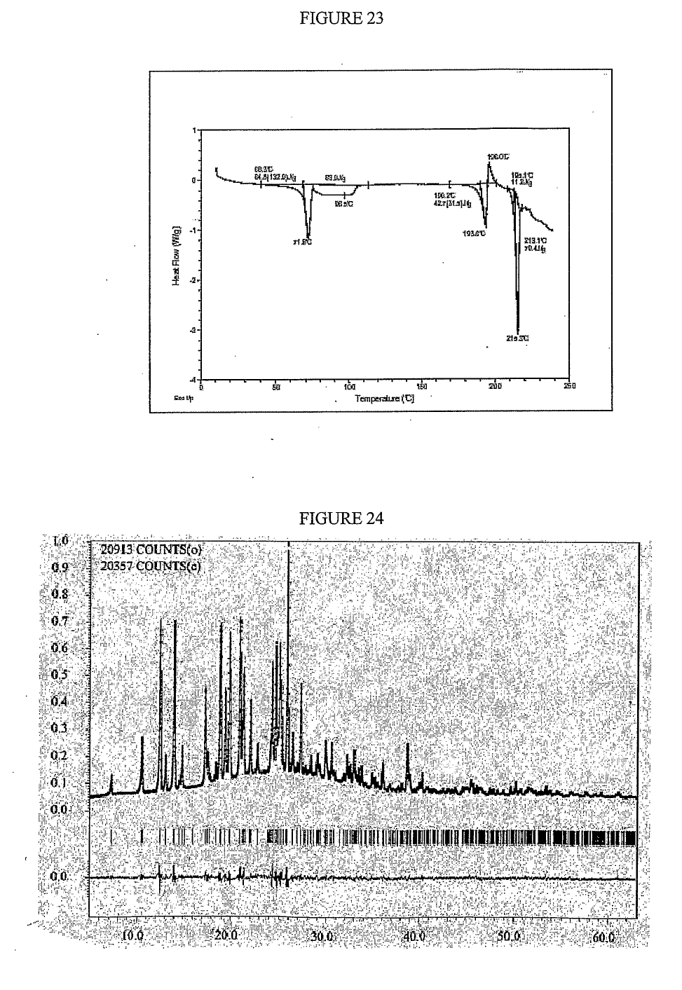

Differential calorimetric analysis of crystal forms I to V was performed on a DSC Q100 (TA Instruments) differential calorimetric analysis instrument. Sensitivity is 0.2 μW in power, 1% in enthalpy and 0.1% in temperature. Samples are placed in capsules crimpered and heated under a nitrogen stream (50 mL/min) at a 5° C./min speed within a temperature interval ranging from 10 to 240° C. Calorimetric events are characterized by the temperature at onset of the event (Tₒₙₛₑₜ) and temperature at its peak (Tₘₐₓ). For each form, the peak corresponding to fusion is measured. Thermic profiles of forms I, II, III, IV and V are represented by FIGS. 19, 20, 21, 22 and 23, respectively.

Results are summarised in table 5.

TABLE 5

| Forms | DSC Tₒₙₛₑₜ/Tₘₐₓ (° C.) |
|---|---|
| I | 183.0/187.3 |
| | 189.8/191.6 (exothermic) |
| | 211.2/214.4 |
| II | 210.1/213.8 |
| III | 169.4/183.4 |
| | 212.9/215.2 |
| IV | 89.0/108.7 |
| | 213.6/215.9 |
| V | 68.3/71.8 |
| | nd*/96.5 |
| | 190.2/193.6 |
| | 195.1/196.0 (exothermic) |
| | 213.1/215.3 |

*nd = non determined

Form I shows two endothermic and one exothermic peak. The first peak corresponds to the fusion of form I (Tₒₙₛₑₜ=183.0° C.). The exothermic peak corresponds to the cristallisation of form II (Tₒₙₛₑₜ=189.8° C.). The third peak corresponds to the fusion of form II (Tₒₙₛₑₜ=211.2° C.).

Analysis of form I at a 40° C./min speed allowed the determination of an enthalphy ΔH of 130.4 J/g.

Form II shows a fusion peak at 210.1° C.

DSC analysis of form III indicates that this form is converted into form II during the heating process.

Form IV presents an endotherm at 108.7° C. that corresponds to the desolvatation of the crystal. The second peak corresponds to fusion of form II (215.9° C.).

DSC analysis of form V indicates a desolvatation in two steps then shows the peaks characterising form I.

6) Stability Studies

Stability of crystal form I compared to that of forms II and III was determined by maturation studies. 100 mg suspensions of the different polymorphic forms, separated or in mixtures, in 2 ml of n-butyl-acetate, were left for maturation during 8 or 72 hours at different temperatures. The result of these experiments is shown in table 6 below.

TABLE 6

| | | | Form(s) identified | | |
|---|---|---|---|---|---|
| | | | after maturation | | |
| | Maturation | | Temperature (° C.) | | |
| Form | time | | 25 | 50 | 80 |
| I | 8 | hours | I | I | I |
| | 3 | days | I | I | I |
| II | 8 | hours | II | II | II |
| | 3 | days | II | II | II |
| III | 8 | hours | I | I | I |
| | 3 | days | I | I | I |
| I + II | 8 | hours | I + II | I + II | I + II |
| | 3 | days | I | I | I |

Form I is stable under the tested conditions. Form II also appears to be a stable form. However, a mixture of forms I and II changes towards form I in all tested conditions. Form III quickly turns into form I.

These different experiments showed that form I is thermodynamically the most stable of the three crystal forms.

The present invention also relates to the processes used for the preparation of crystal form I of besipirdine.HCl.

According to the invention, such a process used to obtain the crystal form I of besipirdine.HCl involves the following steps:
preparation of besipirdine.HCl; as an open-ended example, besipirdine.HCl can be prepared following any known process, including in particular the processes described in U.S. Pat. No. 4,970,218 and WO2005/035496,
besipirdine.HCl is solubilised into a solvent, a mixture of solvents, or a mixture solvent(s)/water, the aforementioned solvents being chosen among the ones in which besipirdine.HCl is soluble,
solvent or mixture is at least partially evaporated,
crystals obtained in that way are collected and dried.

The solvent in which besipirdine.HCl is dissolved is advantageously chosen among polar solvents, alcohols, cetons and esters. Thus, it can be chosen among acetonitrile, acetone, ethanol, ethanol, butanol. As previously mentioned, it can be dissolved in a mixture of these solvents, but also in a mixture of solvent(s), particularly the aforementioned ones, with water; for example, acetonitrile/water and acetone/water mixtures. Proportion of water within these mixtures can vary from 0.01 to 50% in weight of mixture. Thus, acetonitrile/water, 90/10 (v/v) and acetone/water, 90/10 (v/v) mixtures are the preferential mixtures.

According to the invention process, the solvent or the mixture is evaporated at a temperature between 0° C. and the boiling point of the solvent or the mixture. Temperature preferentially lies between 0° C. and room temperature, even between 0° C. and 10° C. At 4° C., evaporation occurs in advantageous conditions.

The aforementioned invention process can be completed with additional steps.

In this way, before or during evaporation, the suspension can be seeded with a low amount of besipirdine.HCl crystal form I in order to favour cristallisation of form I.

The solubilisation step of besipirdine.HCl can be complemented by a solubilisation in the aforementioned solvent or mixture until saturation and, during solvent or mixture evaporation, diffusion of a non-solvent more volatile than the aforementioned solvent or mixture and in which besipirdine.HCl is less soluble than in the aforementioned solvent or mixture.

The non solvent is preferentially diffused at room temperature.

In order to achieve this step, the solvent or mixture and the non solvent are advantageously chosen among the following couples, respectively: acetonitrile and acetone, acetonitrile and hexane, acetonitrile/water (for example in a 90/10 proportion) and cyclohexene, acetonitrile/water (for example in a 90/10 proportion) and acetone, acetone/water (for example in a 90/10 proportion) and cyclohexene, butanol and cyclohexene.

The crystals obtained using this method can be retrieved by filtration after washing.

Another process of the invention for obtaining crystal form I of besipirdine.HCl includes the following steps:
preparation of besipirdine.HCl; as an open-ended example, besipirdine.HCl can be prepared following any known process, including in particular the processes described in U.S. Pat. No. 4,970,218 and WO2005/035496,
obtained besipirdine.HCl is placed and maintained, possibly under agitation, in a humid environment, in which relative humidity is at least 75%, preferentially at least 85%,
crystals obtained in that way are collected and dried.

A humid environment may, for example, be generated by an aqueous solution saturated in potassium nitrate or by a gas flux laden with steam.

The invention also relates to one another process for obtaining crystal form I of besipirdine.HCl; this process includes the following steps:
preparation of besipirdine.HCl; as an open-ended example, besipirdine.HCl can be prepared following any known process, including in particular the processes described in U.S. Pat. No. 4,970,218 and WO2005/035496,
a suspension of besipirdine.HCl is prepared in a solvent in which it is not completely soluble, and this suspension is agitated,
crystals obtained in that way are washed, collected and dried.

Advantageously, before or during evaporation, the suspension is seeded with a low amount of besipirdine.HCl crystal form I.

The retrieved solvent can contain at least water traces. It can be chosen among esters, cetons, ethers and alcohols with at least two carbon atoms. Advantageously, It is chosen among n-butyle acetate, methyl-ethyl-ceton and methyl-isobutyl-ceton.

The maturation step has a variable length, from 5 minutes to one week but is preferentially less than or equal to 24 hours.

The invention also relates to crystal form I of besipirdine.HCl obtained by any of the aforedescribed processes.

Polymorphic form I of besipirdine.HCl is thermodynamically the most stable of all characterized forms under use and storing conditions of the powder. Maturation studies and follow-up of clinical batches of besipirdine.HCl show that a mixture of polymorphic forms changes towards turning into form I. Moreover, polymorphic form I of besipirdine.HCl can be obtained specifically using the process of the invention. This constitutes an advantage for the production of besipirdine.HCl as a form of reasonable pharmaceutical quality.

Thus, polymorphic form I of besipirdine.HCl is particularly suitable for the fabrication of pharmaceutical compositions useful for applications in the treatment of all disorders for which besipirdine is indicated.

In particular, in the case of a prolonged-release formulation, the use of a well characterized and stable polymorphic form will avoid the risk of variation in the dissolution and liberation characteristics of the compound.

From another of its aspects, the present invention relates to pharmaceutical compositions in which besipirdine.HCl as polymorphic form I is the active compound.

Thus, the invention relates to the following purposes:

The use of a crystal form of besipirdine.HCl according to the invention for obtaining a stable form of a pharmaceutical composition. This composition can be a therapeutic composition, which can have an immediate or delayed liberation form.

Since crystal form I of besipirdine.HCl has at least all the therapeutic properties of besipirdine as obtained according to processes of the anterior art, the indications of this specific crystal form are all applications for which besipirdine is indicated. In particular, this form is intended to be used for the treatment of symptoms of bladder irritation associated with indications such as overactive bladder (OAB) or interstitial cystitis, effort incontinence or mixed incontinence.

An advantageous therapeutic composition of the invention contains as the active compound, at least 90% of crystal form I of besipirdine.HCl as previously defined.

Within pharmaceutical compositions of the present invention for oral, sublingual, sub-cutaneous, intramuscular, intravenous, transdermic or local administration, the active compound, alone or in association with another active compound, can be administered as a single entity of administration form, as part of a mixture with classical pharmaceutical media, to animals and humans. The suitable entities of administration forms include the forms to be given per os such as tablets, gelules, pills, granules and solutions or oral suspensions, forms for sublingal and buccal administration, aerosols, implants, forms for local, transdermic, subcutaneous, intramuscular, intravenous, intranasal or intraocular administration.

Within pharmaceutic compositions of the present invention, the active compound or the active compounds are generally formulated in dosage units. One dosage unit contains 0.5 to 300 mg, advantageously 5 to 60 mg and preferentially 5 to 40 mg per dosage unit for daily administrations, one or several times a day.

Although these dosages are examples of intermediate situations, particular cases in which higher or lower dosages are suitable, such dosages are also included in the invention. In usual practice, the dosage appropriate for each patient is determined by the doctor as a function of the mode of administration and the age, weight and response of the aforementioned patient.

When preparing a solid composition as the form of tablets or gelules, a mixture of pharmaceutic excipients made up of diluents such as, for example, lactose, mannitol, microcrystallin cellulose, amidon, dicalcic phosphate, binding agents such as polyvinylpyrrolidone or hydroxypropylmethylcellulose for example, bursting agents such as, for example, crosslinked polyvinylpyrrolidone, crosslinked carboxymethylcellulose, sodium croscarmellose, flowing agents such as silica, talc, lubricants such as magnesium stearate, stearic acid, glycerol tribehenate, sodium stearlyfumarate, is added to the active compounds, micronised or not.

Wetting or tensioactive agents such as sodium laurylsulfate, polysorbate 80, poloxamer 188 can be added to the formulation.

Tablets can be produced using different techniques, direct compression, dry granulation, humid granulation, hot-melt.

Tablets can be nude, sugar-coated (using saccharose for example) or coated with different polymers or other suitable materials.

Tablets can have an immediate, delayed or extended liberation by using polymeric matrices or specific polymers during the coating process.

Gelules can be hard or soft, coated or not, in order to have an immediate, extended or delayed (for example a form for parenteral administration) activity. They can contain not only a solid formulation formulated as previously described for tablets, but also liquids or semi-solids.

A preparation as a syrup or elixir form can contain the active compound or the active compounds together with a sweetener, preferentially acaloric, methylparaben and propylparaben as antiseptic agents as well as a flavouring agent and an appropriate colouring agent.

Water-dispersible powders or granules in water can contain the active compound or the active compounds as a mixture with dispersing or wetting agents, or suspensing agents such as polyvinylpyrrolidone or polyvidone, as well as sweeteners or flavouring agents.

For rectal administration, suppositories are used that are prepared with linking agents melting at rectal temperature, for example cocoa butter or polyethyleneglycols.

For parental, intranasal or intraocular administration, aqueous suspensions, isotonic saline solutions or sterile injectable solutions containing dispersing agents and/or pharmaceutically compatible solubilising agents such as propyleneglycol or butyleneglycol, are used.

Hence, in order to prepare an aqueous solution injectable intravenously, a cosolvant, for example an alcohol such as ethanol or a glycol such as polyethyleneglycol or propyleneglycol, and a hydrophilic tensioactive such as polysorbate 80 or poloxamer 188 can be used. In order to prepare an oileous solution injectable intramuscularly, the active compound can be solubilised using a triglyceride or a glycerol ester.

For local administration, creams, ointments, gels, eye lotions and sprays can be used.

For transdermic administration, patches can be used which can be in multilaminar form or with a reservoir in which the active compound is in alcoholic solution.

For an administration by inhalation, an aerosol containing, for example, sorbitane trioleate or oleic acid as well as trichlorofluoromethan, dichlorofluoromethan, dichlorotetra-fluoroethan, freon substitutes or any other biologically compatible propulsing gas is used; a system containing the active compound, alone or associated with an excipient, all as powders, can be used.

The active compound or the active compounds can also be presented as a complex with a cyclodextrin, for example α-, β- or γ-cyclodextrin, 2-hydroxypropyl-β-cyclodextrin or methyl-β-cyclodextrin.

The active compound or the active compounds can also be formulated as microcapsules or microspheres, possibly with one or several carriers or additives.

Among the extended liberation forms useful for chronic treatments, implants can be used. These implants can be prepared as an oileous suspension or a suspension of microspheres in an isotonic environment.

Preferentially, besipirdine.HCl as crystal form I is administered per os, once daily.

From another angle, the invention also relates to a method involving the administration of a therapeutically effective amount of besipirdine.HCl as polymorph I.

The following examples show several methods of obtention of form I, except example 1, which indicates the method of obtention of other forms of besipirdine.HCl.

Examples 3 to 11 illustrate methods of cristallisation allowing the obtention of monocrystals. In examples 6 to 11, the vapour diffusion technique is used: a solution saturated in compound in a relatively non-volatile solvent, is placed in a small container. This container is placed into a dessicator containing a solvent more volatile than the one in which besipirdine.HCl is not soluble. The vapour of this solvent diffuses slowly into the container, favouring the precipitation of the compound as unique crystals (X-ray Structure Determination A Practical Guide, 2nd edition, George H. Stout and Lyle H. Jensen, John Wiley & Sons, New York, 1989). Characterisation of the crystals is preformed by optical microscopy and DSC.

Example 12 shows a method of obtention of form I by maturation in a humid environment, without any recristallisation step.

Examples 13 to 18 present methods of production in which transformation is achieved by maturation in suspension (slurry transformation).

### EXAMPLE 1

### Obtention of Crystal Forms II, II, IV and V from besipirinde.HCl Synthesised According to the Process Described in Patent Application WO 2005/035496

Characterisation of the samples is performed by DSC, TG and PXRD.

In order to obtain the polymorphic form II of besipirdine.HCl, 200 mg of powder are heated at 200° C. until complete melt, then it is recristallised at 25° C. for 10 days. No solvent restraint is observed.

Polymorphic form III is obtained by solubilising 200 mg of powder in a 6 ml volume of acetonitrile at 70° C. under agitation, followed by the evaporation of the solvent at 25° C. in a dessicator for 8 days. No solvent restraint is observed.

Solvate form IV is obtained by solubilising 200 mg of powder in a 4 ml volume of methanol at room temperature, followed by the evaporation of the solvent at 4° C. in a dessicator for 7 days.

Solvate form V is obtained by solubilising 200 mg of powder in a 4 ml volume of ethanol at room temperature, followed by the evaporation of the solvent at 4° C. in a dessicator for 7 days.

### EXAMPLE 2

### Obtention of crystal form I in a mixture of 90% Acetonitrile and 10% Water Mixture

200 mg of besipirdine.HCl in 1 ml acetonitrile/water (90/10: v/v) mixture are solubilised at room temperature under agitation. Solvent is evaporated at 4° C. in a dessicator for 8 days.

### EXAMPLE 3

### Obtention of Crystal form I in Acetonitrile

A solution saturated in besipirdine.HCl in acetonitrile is prepared at room temperature and under agitation. Solvent is evaporated at 4° C. Crystals are dried in a dessicator then characterized by DSC and optical microscopy. The crystals that are formed look like white needles.

### EXAMPLE 4

### Obtention of Crystal form I in Ethanol

A solution saturated in besipirdine.HCl in ethanol is prepared at room temperature and under agitation. Solvent is evaporated at 4° C. Crystals are dried in a dessicator then characterized by DSC and optical microscopy. The crystals that are formed look like a mixture of white prisms and beige blocks.

### EXAMPLE 5

### Obtention of Crystal Form I in a Mixture of 90% Acetone and 10% Water Mixture

200 mg of besipirdine.HCl in 1 ml acetone/water (90/10: v/v) mixture are solubilised at room temperature under agitation. Solvent is evaporated at 4° C. in a dessicator for 8 days. Crystals are dried in a dessicator then characterized by DSC and optical microscopy. The crystals that are formed look like beige sheets.

### EXAMPLE 6

### Obtention of Crystal Form I in Acetonitrile Using the Vapour Diffusion Method

A solution saturated in besipirdine.HCl in acetonitrile is prepared at room temperature and under agitation. Sample is placed in a dessicator at room temperature in an environment rich in acetone to favour precipitation. Crystals are dried in a dessicator then characterized by DSC and optical microscopy. The crystals that are formed look like beige sheets and microcrystals.

### EXAMPLE 7

### Obtention of Crystal Form I in Acetonitrile Using the Vapour Diffusion Method

A solution saturated in besipirdine.HCl in acetonitrile is prepared at room temperature and under agitation. Sample is placed in a dessicator at room temperature in an environment rich in hexan to favour precipitation. Crystals are dried in a dessicator then characterized by DSC and optical microscopy. The crystals that are formed look like beige sheets.

### EXAMPLE 8

### Obtention of Crystal Form I in a Mixture of 90% Acetonitrile and 10% Water Mixture Using the Vapour Diffusion Method

A solution saturated in besipirdine.HCl in an acetonitrile/water (90/10: v/v) mixture is prepared at room temperature and under agitation. Sample is placed in a dessicator at 4° C. whose air is rich in cyclohexen to favour precipitation. Crystals are dried in a dessicator then characterized by DSC and optical microscopy. The crystals that are formed look like beige sheets.

### EXAMPLE 9

### Obtention of Crystal Form I in a Mixture of 90% Acetonitrile and 10% Water Using the Vapour Diffusion Method

A solution saturated in besipirdine.HCl in an acetonitrile/water (90/10: v/v) mixture is prepared at room temperature and under agitation. Sample is placed in a dessicator at room temperature whose air is rich in acetone to favour precipitation. Crystals are dried in a dessicator then characterized by DSC and optical microscopy. The crystals that are formed look like white stars.

### EXAMPLE 10

### Obtention of Crystal Form I in a Mixture of 90% Acetone and 10% Water Using the Vapour Diffusion Method

A solution saturated in besipirdine.HCl in an acetone/water (90/10: v/v) mixture is prepared at room temperature and under agitation. Sample is placed in a dessicator at 4° C. whose air is rich in cyclohexen to favour precipitation. Crystals are dried in a dessicator then characterized by DSC and optical microscopy. The crystals that are formed look like beige sheets.

### EXAMPLE 11

### Obtention of Crystal Form I in Butanol Using the Vapour Diffusion Method

A solution saturated in besipirdine.HCl in butanol is prepared at room temperature and under agitation. Sample is placed in a dessicator at 4° C. whose air is rich in cyclohexen to favour precipitation. Crystals are dried in a dessicator then characterized by DSC and optical microscopy. The crystals that are formed look like beige sheets.

### EXAMPLE 12

### Obtention of Crystal Form I by Transformation in Solid Phase (Maturation in a Humid Atmosphere)

100 mg of besipirdine.HCl as crystal form III or as a mixture of forms II and III are placed overnight in a closed dessicator whose lowest part is filled with a solution saturated in potassium nitrate. Such conditions induce an atmosphere with about 85% relative humidity. Samples are then placed in a standard dessicator Drierite to provide form I.

### EXAMPLE 13

### Obtention of Crystal Form I by Transformation in Solid Phase (Maturation in a Humid Atmosphere)

20 g of besipirdine.HCl as a mixture of forms II and III are placed in a 200 ml evaporation balloon flask. This balloon flask is placed on a rotative evaporator whose lowest part is charged with an aqueous saturated solution of potassium nitrate. The whole thing is closed so that the relative humidity reaches an equilibrium at about 85%, with a rotation speed of about 30 turns/min. The balloon flask is regularly weighted and samples are taken up for differential calorimetry analysis. After 4 days, the DSC analysis, confirmed by the X-ray powder diagram, indicates that transformation into form I is total. HPLC analysis indicates a purity of 99.95%, identical to that of the original material.

### EXAMPLE 14

### Obtention of Crystal Form I by Transformation in methyl-isobutyl-cetone (Slurry Transformation)

100 mg of besipirdine.HCl as a mixture of forms II and III is mixed with methyl-isobutyl-cetone containing water traces for about 24 hours. Crystals are then isolated, dried under nitrogen and characterized by DSC and optical microscopy. DSC analysis indicates the transitions associated with form I.

### EXAMPLE 15

### Obtention of Crystal Form I by Transformation in ethylmethylcetone (Slurry Transformation)

1 g of besipirdine.HCl as a mixture of forms II and III is mixed overnight, at room temperature, with 3 ml of ethylmethylcetone containing 2 μl of water. The mixture is then filtered, washed with methyl-ethyl-cetone and vacuum-dried for 3 hours. About 0.9 g of besipirdine.HCl as form I are obtained.

The compound is characterized by DSC and optical microscopy. DSC analysis indicates the transitions associated with form I.

### EXAMPLE 16

### Obtention of Crystal Form I by Transformation in n-butyle Acetate (Slurry Transformation)

1 g of besipirdine.HCl as a mixture of forms II and III is mixed overnight, at room temperature, with 3 ml of n-butyle acetate saturated in water (about 1%). The mixture is then filtered, washed with n-butyle acetate and vacuum-dried for 3 hours. About 0.9 g of besipirdine.HCl as form I are obtained.

The compound is characterized by DSC and optical microscopy. DSC analysis indicates the transitions associated with form I.

### EXAMPLE 17

### Obtention of Crystal Form I by Transformation in Water-Saturated n-butyle Acetate (Slurry Transformation)

20 g of besipirdine.HCl as a mixture of forms II and III are suspended in 100 ml water-saturated n-butyle acetate. The obtained suspension is mixed under a nitrogen atmosphere for 24 hours at room temperature. The solution is then filtered then washed 3 times with 20 ml of pure undiluted n-butyle acetate. After 30 min air-drying, the white solids are vacuum-dried overnight at 25° C. to eliminate residual solvent. The efficiency of the operation is 97%. HPLC analysis indicates a purity >99.97%. Transformation into form I is confirmed by DSC and PXRD analyses.

### EXAMPLE 18

### Preparation of an Immediate Release Form from Polymorphic Form I of besipirdine.HCl

From polymorphic form I of besipirdine.HCl, immediate release gelules are prepared by granulation in humid phase using the composition indicated in the table below:

| Ingredient | Amount (mg) |
|---|---|
| Besipirdine HCl, form I | 20.00 |
| Amidon (corn) | 95.75 |
| Pregelatinised amidon (starch 1500) | 35.00 |
| Sodic croscarmellose | 12.00 |
| Microcristallin cellulose | 80.00 |
| Magnesium stearate | 1.50 |
| Colloidal silicon dioxide | 0.75 |
| Purified water qsf | |
| Total | 245.00 |

Corn amidon and besipirdine.HCl are introduced in the granulator and mixed for about 5 minutes.

Microcristallin cellulose, pregelatinised amidon and a proportion (50%) of sodic croscarmellose are added. The whole ingredients are mixed for about 5 minutes. Granulation of the powder is performed by adding demineralised water (39% w/w) with a 15 ml/min flow, until obtention of a density of between 0.45 and 0.5 g/cm³. Granules are dried on a fluidised bed at 60° C. for 30 minutes until obtention of a residual humidity ratio below 5%.

Dried granules are calibrated on a 630 μm sieve, introduced in a container with the remainder of sodium croscarmellose and mixed for 5 minutes. Magnesium stearate and colloidal silicon dioxide are then added and mixed for 15 minutes.

Size 1 gelatin gelules are manually filled with the final mixture.

### EXAMPLE 19

### Preparation of an Immediate Release Form from Polymorphic Form I of besipirdine.HCl

From polymorphic form I of besipirdine.HCl, immediate release tablets are prepared that have the composition indicated in the table below:

| Ingredient | Amount (mg) |
|---|---|
| Besipirdine HCl, form I | 20.00 |
| Microcristalline cellulose | 174.00 |
| Aerosil | 1.00 |
| Sodic croscarmellose | 4.00 |
| Magnesium stearate | 1.00 |
| Total | 200.00 |

All ingredients, except magnesium stearate, are mixed in a Turbula mixer for 10 minutes. Magnesium stearate is then added and mixed for 5 minutes. Tablets are obtained by direct compression using a rotative press.

1. Crystal form of besipirdine.HCl (Form I) corresponding to the formula A below: 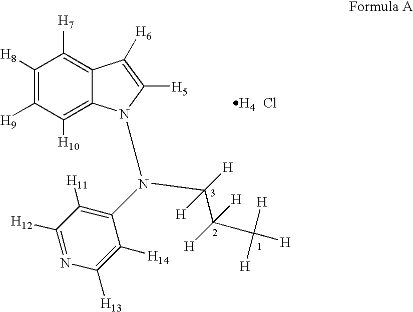
the aforementioned form being characterized by at least one of the following physico-chemical properties:
a) in FTIR, it displays at least the following absorption bands of the infrared spectrum: 778, 1198, 1121, but not the following absorption bands of the infrared spectrum: 3395, 1583, 732, the aforementioned bands being expressed in cm⁻¹ at ±5 cm⁻¹;
b) in PXRD, it shows at least the following reflections, which are the most intense ones but whose intensity hereafter is given for information only:

| | Angle (2θ) | | | | | | |
|---|---|---|---|---|---|---|---|
| | 12.61 | 14.11 | 18.98 | 19.93 | 21.03 | 25.13 | 25.91 |
| Intensity (%) | 77.13 | 71.82 | 73.00 | 67.21 | 61.08 | 60.44 | 100.00 |

c) in DSC, it displays at least an endothermic peak at 187.3±2.0° C. using 5° C./min scanning conditions, and a fusion enthalpy ΔH of 130.4±2.0 J/g.

2. Crystal form according to claim 1, characterized by at least two of the characteristics a), b) and c).

3. Crystal form according to claim 1, characterized by three of the characteristics a), b) and c).

4. Process for obtaining crystal form I of besipirdine.HCl, as defined in claim 1, comprising:
preparation of besipirdine.HCl,
solubilisation of besipirdine.HCl in a solvent, a mixture of solvents or a mixture of solvent(s)/water, the aforementioned solvent(s) being chosen among those in which besipirdine.HCl is soluble,
evaporation, at least partial, of solvent or mixture, and
retrieval and drying of obtained crystals.

5. Process according to claim 4, wherein besipirdine.HCl is solubilised in a solvent chosen among polar solvents, alcohols, cetones and esters.

6. Process according to claim 5, wherein the solvent is chosen among acetonitrile, acetone, ethanol, butanol.

7. Process according to claim 4, wherein the mixture of solvent(s) with water is chosen among acetonitrile/water and acetone/water mixtures.

8. Process according to claim 7, wherein the mixture of solvent(s) with water is chosen among acetonitrile/water, 90/10 (v/v) and acetone/water, 90/10 (v/v) mixtures.

9. Process according to claim 4, wherein the solvent or the mixture is evaporated at a temperature between 0° C. and room temperature.

10. Process according to claim 9, wherein evaporation is performed between 0° C. and 10° C.

11. Process according to claim 10, wherein evaporation is performed at a temperature of about 4° C.

12. Process according to claim 4, wherein, before or during evaporation, suspension is seeded with a low amount of crystal form I of besipirdine.HCl.

13. Process according to claim 4, wherein besipirdine.HCl is solubilised in the solvent or mixture until saturation, and, during evaporation of the solvent or mixture, a non solvent more volatile than the solvent or mixture and in which besipirdine.HCl is less soluble than in the solvent or mixture, is diffused during evaporation of the solvent or mixture.

14. Process according to claim 13, wherein the non-solvent is diffused at room temperature.

15. Process according to claim 13, wherein the solvent or mixture and the non-solvent are chosen among one of the following couples: acetonitrile and acetone, acetonitrile and hexane, acetonitrile/water and cyclohexene, acetonitrile/water and acetone, acetone/water and cyclohexene, butanol and cyclohexene.

16. Process according to claim 4, wherein crystals are retrieved by filtration.

17. Process for obtaining crystal form I of besipirdine.HCl, comprising:
preparation of besipirdine.HCl,
maintaining obtained besipirdine.HCl in a humid environment in which relative humidity is at least 75%, and
retrieval and drying of obtained crystals.

18. Process according to claim 17, wherein relative humidity is at least 85%.

19. Process according to claim 17, wherein the humid atmosphere is generated by an aqueous solution saturated in potassium nitrate.

20. Process for obtaining crystal form I of besipirdine.HCl, comprising:
preparation of besipirdine.HCl,
preparation of a suspension of besipirdine.HCl in a solvent in which it is not completely soluble, and agitation of this suspension, and
washing, retrieval and drying of obtained crystals.

21. Process according to claim 20, wherein, before or during evaporation, the suspension is seeded with a low amount of crystal form I of besipirdine.HCl.

22. Process according to claim 21, wherein the solvent contains water traces, at least.

23. Process according to claim 20, wherein the said solvent is chosen among esters, cetones, ethers and alcohols including at least two carbon atoms.

24. Process according to claim 20, wherein the solvent is chosen among n-butyle acetate, methyl-ethyl-cetone and methyl-isobutyl-cetone.

25. Process according to claim 17, wherein the suspension is agitated for at least one day.

26. Crystal form of besipirdine.HCl corresponding to formula A below: 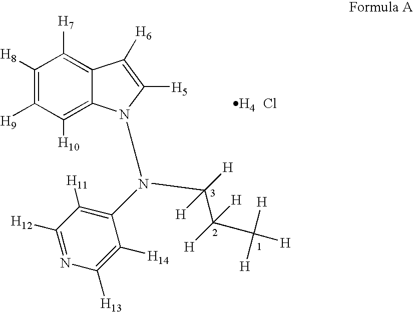
which is obtained by a process according to claim 4.

27. A method for obtaining a stable form of a pharmaceutical composition utilizing crystal form I of besipirdine.HCl according to claim 1.

28. The method of claim 27, wherein the pharmaceutical composition is a therapeutic composition.

29. The method of claim 28, wherein the pharmaceutical composition is under a form with immediate or delayed liberation.

30. The method of claim 28, wherein the therapeutic composition is intended to the treatment of symptoms of bladder irritation associated with indications such as overactive bladder (OAB) or interstitial cystitis, or to the treatment of stress urinary incontinence or mixed incontinence.

31. Therapeutic composition containing as the active compound, at least 90% of crystal form I of besipirdine.HCl as defined claim 1.
